(19) United States
(12) Patent Application Publication
(10) Pub. No.: US 2023/0000806 A1
(43) Pub. Date: Jan. 5, 2023

(54) COMPOSITIONS AND METHODS FOR GLUTATHIONE ENHANCEMENT FOR USE IN BRAIN HEALTH

(71) Applicant: SOCIETE DES PRODUITS NESTLE S.A., Vevey (CH)

(72) Inventors: CARMEN SANDI, Bremblens (CH); ALINA STRASSER, Dusseldorf (DE); PASCAL STEINER, St. Louis, MO (US); NICOLAS PREITNER, Lausanne (CH); LAURA TROVO, Paudex (CH)

(21) Appl. No.: 17/756,090

(22) PCT Filed: Nov. 16, 2020
(86) PCT No.: PCT/EP2020/082191

§ 371 Date: May 17, 2022

(30) Foreign Application Priority Data
Nov. 18, 2019 (EP) 19209825.9

(51) Int. Cl. A61K 31/198; A61K 35/20; A61K 31/205; A61K 31/4172; A61P 25/22; A61K 9/00

(52) U.S. Cl. CPC A61K 31/198; A61K 35/20; A61K 31/205; A61K 31/4172; A61P 25/22; A61K 9/0053

(57) ABSTRACT

The present invention relates to compositions and methods for potentiating glutathione enhancement for use in brain health. In particular, the compositions and methods of the invention are beneficial for use in subjects in need of increasing motivational performance and/or mental energy, functions that may be decreased upon stress and anxiety.

### FIELD OF THE INVENTION

The present invention relates to compositions and methods for potentiating glutathione enhancement for use in brain health. In particular, the compositions and methods of the invention are beneficial for use in subjects in need of increasing motivational performance and/or mental energy, functions that may be decreased upon stress and anxiety.

### BACKGROUND TO THE INVENTION

Glutathione (GSH) is an essential antioxidant used by the body to prevent cellular and tissue damage. It is involved in many fundamental metabolic processes ranging from the nitric oxide cycle to dietary mineral incorporation. Additionally, glutathione is instrumental for cells to regulate their division and their differentiation from progenitor cells into mature somatic cells.

As one of the body's core antioxidants, glutathione binds circulating reactive oxygen species (ROS) which can cause cellular and DNA damage if left unchecked. Reactive oxygen species, also known as free radicals, are byproducts of metabolism and can be broadly harmful to the body. To scavenge circulating ROS, glutathione binds to ROS thereby becoming oxidized. This means that glutathione prevents important cellular proteins or DNA from being oxidized, which can inhibit their function.

Increased oxidative damage and decreased glutathione levels are observed under situations of high energetic demand in the brain such as psychogenic stress.

High concentrations of oxidized glutathione in the brain are a hallmark that the brain is in a compromised state, but high concentrations in the blood plasma may be considered to be healthy and normal. The reason is that oxidized glutathione must return to the bloodstream from the brain in order to discharge the ROS it carries into a metabolic processes which can make use of them constructively. Alternatively, the oxidized form of GSH can be locally reverted back into the reduced state by glutathione reductase or it can return from the brain to the bloodstream in order to discharge the ROS constructively. As such, high concentrations of oxidized glutathione in the brain may mean that there is not enough glutathione to remove all of the reactive oxygen species that are circulating, indicating severe levels of stress.

By only measuring glutathione levels in blood plasma, one may erroneously assume that circulating glutathione is normal, even in cognitively impaired individuals. Only recently, it was recognized that cognitively impaired individuals have decreased glutathione levels in the brain, however, it is not known under what conditions glutathione levels in the brain may transiently change in normal healthy individuals related to their performance of different cognitive and motor tasks.

Direct supplementation with GSH is a challenging approach, particularly because it can be rapidly degraded in the liver into its constituent amino acids as well as be partially hydrolyzed and oxidized. Consequently, GSH bioavailability is limited following oral administration. Therefore, there is a need to find compositions and methods of increasing glutathione in the brain in situations of high energetic demand in the brain.

### SUMMARY OF THE INVENTION

The present invention provides compositions and methods for enhancement of glutathione levels in the brain. In particular, the present invention provides compositions and methods for enhancement of glutathione during high energy demands in the brain, for example, to be used to increase motivational performance and/or mental energy.

As mentioned above, direct oral administration of glutathione has limited bioavailable effectiveness. The present invention provides solutions for enhancement of glutathione in the brain, particularly in the nucleus accumbens region, during high energy demands in the brain by providing:
(i) compounds which are substrates or precursors of substrates of glutathione synthesis;
(ii) compounds which are targeting the regulation of anti-oxidant expression levels of the enzymes involved in its synthesis, for example, by targeting the main regulator of the antioxidant response system, Nrf2-dependent transcription and/or
(iii) compounds which further potentiate the antioxidant effect of glutathione, such as additional antioxidants.

In several embodiments, compounds which are precursors of glutathione, such as glycine, cysteine or glutamate or their functional derivatives may be administered to increase the glutathione in the brain. Other substrates or precursors of substrates involved in the synthesis of glutathione are, for example, N-Acetylcysteine, taurine, and whey proteins.

In several embodiments, compounds which are targeting regulation of antioxidant expression via Nrf2 may be administered to increase the glutathione in the brain. For example, sulforaphane, dimethylfumarate, curcumin, melatonin, and trehalose.

In several embodiments, compounds which are antioxidants influencing glutathione may be administered to increase the glutathione in the brain. For example, puerarine, ergothioneine, l-carnitine, L-theanine, and glutamine.

In a preferred embodiment, the composition of the invention comprises a selection of at least one compound from each category of:

(i) compounds which are substrates or precursors of substrates of glutathione synthesis;

(ii) compounds which are targeting the regulation of anti-oxidant expression levels of the enzymes involved in its synthesis, for example, by targeting the main regulator of the antioxidant response system, Nrf2-dependent transcription and/or

(iii) compounds which further potentiate the antioxidant effect of glutathione, such as additional antioxidants.

The present inventions provides methods and uses of these substances for enhancement of glutathione in the brain, in particular in the form of a “food,” “beverage”, “food product”, “beverage product”, “food composition” and “beverage composition” which is a product or composition that is intended for ingestion by an individual.

In particular, the compositions and methods of the invention are beneficial for use in subjects in need of increasing motivational performance and/or mental energy.

### DESCRIPTION OF FIGURES

The following abbreviations refer respectively to the definitions below: GSH (Glutathione); TAU (Taurine); HA (High Anxious); LA (Low Anxious); NAc (Nucleus Accumbens); VS (ventral striatum); 1H MRS (proton magnetic resonance spectroscopy); MID (monetary incentive delay); Cort (cortisol).

FIG. 1—GSH concentrations measured in the nucleus accumbens positively correlates with performance

FIG. 1 shows that GSH concentrations measured in the nucleus accumbens by 1H MRS significantly correlates with total performance in a subsequent monetized hand grip effort task. (A) GSH positively correlates with performance across all trial blocks. (B) GSH positively correlates with performance in individual trial blocks. GSH (Glutathione); 1H MRS (proton magnetic resonance spectroscopy)

FIG. 2—GSH concentration measured in the nucleus accumbens negatively correlated with cortisol levels

FIG. 2 shows that GSH concentrations measured in the nucleus accumbens by 1H MRS are negatively correlated with changes in cortisol levels sampled during a hand grip effort task.

FIG. 3—Classification of high or low anxious behaviour

FIG. 3 shows how inbred mice can be classified as either high or low anxious according to their behaviour in tests of anxiety-like behaviour. (A) High anxious mice spend significantly less time in the open arms of an elevated plus maze. (B) High anxious mice spend significantly less time in the anxiogenic lit compartment of a light-dark box.

FIG. 4—Low anxiety correlated with higher GSH in nucleus accumbens

FIG. 4 shows how mice characterized for their natural trait anxiety in an elevated plus maze exhibit significant differences in GSH concentrations in the nucleus accumbens under basal conditions.

FIG. 5—Cytoplasmic Reactive Oxygen Species Measurements for N-Acetylcysteine

FIG. 5A shows the result of N-Acetylcysteine at baseline and FIG. 5B after oxidative stress.

FIG. 6—Cytoplasmic Reactive Oxygen Species Measurements for Puerarine

FIG. 6A shows the result of Puerarine at baseline and FIG. 6B after oxidative stress.

FIG. 7—Cytoplasmic Reactive Oxygen Species Measurements for Sulfurophane

FIG. 7A shows the result of Sulfurophane at baseline and FIG. 7B after oxidative stress.

FIG. 8—Cytoplasmic Reactive Oxygen Species Measurements for Taurine

FIG. 8A shows the result of Taurine at baseline and FIG. 8B after oxidative stress.

FIG. 9—Cytoplasmic Reactive Oxygen Species Measurements for Ergothionine

FIG. 9A shows the result of Ergothionine at baseline and FIG. 9B after oxidative stress.

FIG. 10—Cytoplasmic Reactive Oxygen Species Measurements for L-Theanine

FIG. 10A shows the result of L-Theanine at baseline and FIG. 10B after oxidative stress.

FIG. 11—Nrf2 activation in astrocyte cells by N-Acetylcysteine

FIG. 11 shows that N-Acetylcysteine does not activate Nrf2 hence having no effect on glutathione genes. This supports N-Acetylcysteine as a precursor to GSH but not an Nrf2 activator.

FIG. 12—Nrf2 activation in astrocyte cells by Puerarine

FIG. 12 shows that Puerarine significantly activates Nrf2 at the highest dose of 10 μm with up to a 20% increase Nrf2 levels in the cell nucleus.

FIG. 13—Nrf2 activation in astrocyte cells by Sulfurophane

FIG. 13 shows that Sulfurophane significantly activates Nrf2 at 2 μM with up to a 20% increase Nrf2 levels in the cell nucleus.

FIG. 14—Nrf2 activation in astrocyte cells by Taurine

FIG. 14 shows that Taurine does not activate Nrf2 thus having no effects on downstream GSH genes.

FIG. 15—Nrf2 activation in astrocyte cells by Ergothioneine

FIG. 15 shows that Ergothioneine significantly activates Nrf2 at 0.5 mM with up to an 18% increase Nrf2 levels in the cell nucleus.

FIG. 16—N-Acetylcysteine and Intracellular GSH

N-Acetylcysteine does not increase intracellular glutathione due to the culture conditions (no cysteine depletion)

FIG. 17—Puerarine and Intracellular GSH

Puerarine increases intracellular glutathione

FIG. 18—Sulfurophane and Intracellular GSH

Sulfurophane increases intracellular glutathione.

FIG. 19—Taurine and Intracellular GSH

Taurine increases intracellular GSH.

FIG. 20—Ergothionine and intracellular GSH

Ergothionine increases intracellular GSH.

FIG. 21—L-Theanine and intracellular GSH

L-Theanine increases intracellular GSH

FIG. 22—Enhancement of GSH via systemic treatment with N-acetyl cysteine (NAC) results in increased motivational performance in adult male rats

(A) Schematic for training schedule—Rats were trained to nose poke for saccharine pellets on an FR1 schedule (1 nose poke gives 1 pellet). They were then treated with either NAC (N-Acetylcysteine) or vehicle for 2 weeks in the drinking water. They were given a reminder training session and then 24 h later tested for their motivated effort in a progressive ratio task.

(B) NAC treated rats showed enhanced GSH in the nucleus accumbens compared to vehicle treated rats

(C) NAC treated rats made significantly more nose pokes for the reward than vehicle treated rats

(D) NAC treated rats earned more overall rewards than vehicle treated rats

(E) NAC treated rats exhibited a higher breakpoint than vehicle treated rats.

FIG. 23—N-acetylcysteine and L-cysteine in medium reduced in cysteine and methionine

N-acetylcysteine and L-cysteine significantly increase the GSH intracellular level.

FIG. 24—BSO versus Vehicle on GSH levels

BSO reduced GSH levels by 29% in the Nucleus Accumbens compared to the Vehicle.

FIG. 25—BSO versus Vehicle on Correct Nose Pokes

Decreased performance of the BSO-treated compared to the vehicle-treated animals in the operant conditioning paradigm (PR-schedule) was seen by a significant reduction in correct nose pokes (−60%).

FIG. 26—BSO versus Vehicle on Rewards

Decreased performance of the BSO-treated compared to the vehicle-treated animals in the operant conditioning paradigm (PR-schedule) was seen by a significant reduction in rewards.

FIG. 27—BSO versus Vehicle on Breakpoint

Decreased performance of the BSO-treated compared to the vehicle-treated animals in the operant conditioning paradigm (PR-schedule). This was seen by a significant reduction in breakpoint (−68%).

### DETAILED DESCRIPTION OF THE INVENTION

### Definitions

All percentages are by weight of the total weight of the composition unless expressed otherwise. Similarly, all amounts and all ratios are by weight unless expressed otherwise. When reference is made to the pH, values correspond to pH measured at 25° C. with standard equipment. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms.

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

The “subject” or “individual” of the present invention is an human adult subject, preferably a healthy adult with the need to improve motivational performance through modulating glutathione levels in the brain. The compositions of the invention may be beneficially used for increasing glutathione level in the brain, in particular, the nucleus accumbens for preventing or treating conditions or diseases which are characterized by low glutathione levels in the brain, whether transient or chronic.

In biology and psychology, the term “stress” refers to the consequence of the failure of a human or other animal to respond appropriately to physiological, emotional, or physical threats, whether actual or imagined. The psychobiological features of stress may present as manifestations of oxidative stress, i.e., an imbalance between the production and manifestation of reactive oxygen species and the ability of a biological system readily to detoxify the reactive intermediates or to repair the resulting damage. Disturbances in the normal redox state of tissues can cause toxic effects through the production of peroxides and free radicals that damage all of the components of the cell, including proteins, lipids, and DNA. Some reactive oxidative species can even act as messengers through a phenomenon called “redox signaling.”

“Reactive oxygen species” play important roles in cell signaling, a process termed redox signaling. Thus, to maintain proper cellular homeostasis a balance must be struck between reactive oxygen production and consumption. One source of reactive oxygen under normal conditions in humans is the leakage of activated oxygen from mitochondria during oxidative phosphorylation. Other enzymes capable of producing superoxide (O2-) are xanthine oxidase, NADPH oxidases and cytochromes P450. Hydrogen peroxide, another strong oxidizing agent, is produced by a wide variety of enzymes including several oxidases.

The terms “treatment” and “treating” include any effect that results in the improvement of the condition or disorder, for example lessening, reducing, modulating, or eliminating the condition or disorder. The term does not necessarily imply that a subject is treated until total recovery. Non-limiting examples of “treating” or “treatment of” a condition or disorder include: (1) inhibiting the condition or disorder, i.e., arresting the development of the condition or disorder or its clinical symptoms and (2) relieving the condition or disorder, i.e., causing the temporary or permanent regression of the condition or disorder or its clinical symptoms. A treatment can be patient- or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. The terms “condition” and “disorder” mean any disease, condition, symptom, or indication.

The relative terms “improved,” “increased,” “enhanced” and the like refer to the effects of the composition on increasing glutathione in the brain, in particular in the nucleus accumbens region of the brain, and subsequently improving the cognitive or motor performance in the individual subject.

“Motivational performance” is synonymous with the terms “mental energy” and related terms of “volition”, “will-power”, “time-on-task”, “persistence”, “self-control”, “sustained effort”, and “self-efficacy”. All these terms relate to a person's drive to initiate and do things. Motivational performance is linked to subjectively perceived self-efficacy and well-being.

Motivational performance describes the subjective perception of mental resources available, which in turn is linked to cognitive functioning (Egan et al. (2015) Personality & Social Psychology Bulletin, 41(3), 336-350). For example, motivational performance is reduced in states of depression and anxiety (O'Connor et al. (2006) Nutrition Reviews, 64(7 Pt 2), S2-6).

Measurement of “motivational performance” can be by both motor tasks and cognitive tasks. Typically, these motor tasks and cognitive tasks are performed under incentivized conditions, meaning that individuals get an incentive depending on their performance of the task.

For example, a motor task under incentivized conditions may be measured as an individual's ability to perform a strenuous motor task, e.g. squeezing a handgrip measuring both force and endurance wherein the performance is normalised for individual muscular strength (Zhu et al. (2019) NeuroImage. Clinical, 23, 101922).

For example, a cognitive task under incentivized conditions may be an individual's ability to perform a strenuous cognitive task, e.g. continuous/sustained attention and working memory (e.g. Unsworth et al. (2019) Journal of Experimental Psychology. Learning, Memory, and Cognition), mental arithmetic, or spatial reasoning (e.g. Nagase et al. (2015) Journal of the Society for Neuroscience, 38(10), 2631-2651) wherein the performance is normalised for individual capacity to perform this task.

In animals, such as rodents, measurement of “motivational performance” is measured through motor tasks such as the forced swim test or cognitive tasks such as social dominance test or operant conditioning. Motivational performance can also be measured in relation to anxiety in tests such as “elevated plus maze” (e.g. Hollis et al. (2018) Neuropharmacology, 138, 245-256) and “open field and novel object” (e.g. Toledo-Rodgriguez and Sandi, (2011) Frontiers in Behavioral Neuroscience, 5, 17).

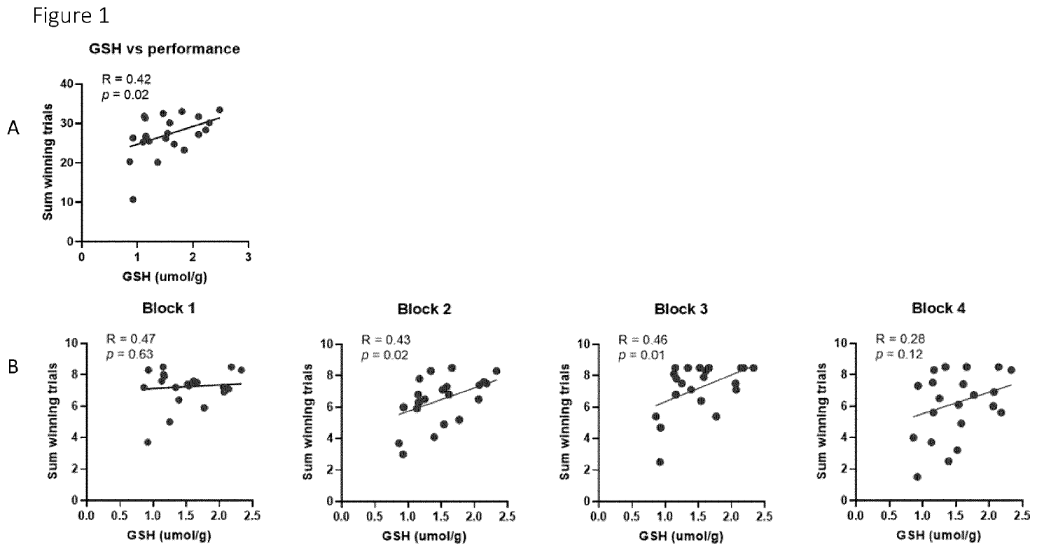

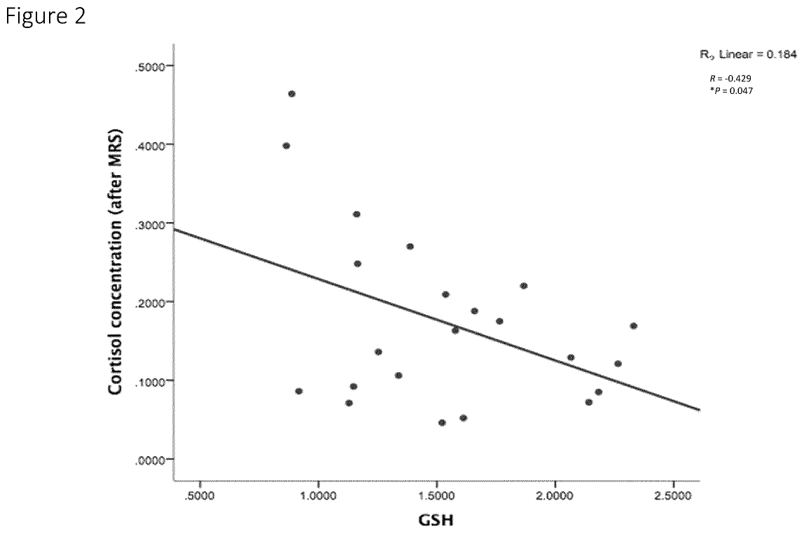

The “nucleus accumbens” is the most ventral part of the striatum and is mainly connected to the limbic system. As a functionally central structure between amygdala, basal ganglia, mesolimbic dopaminergic regions, mediodorsal thalamus and prefrontal cortex, the nucleus accumbens appears to play a modulative role in the flow of the information from the amygdaloid complex to these regions. Together with the prefrontal cortex and amygdala, nucleus accumbens consists a part of the cerebral circuit which regulates functions associated with effort or motivated performance. It is anatomically located in a unique way to serve emotional and behavioral components of feelings. It is considered as a neural interface between motivation and action, having a key-role in food intake, sexual behavior, reward-motivated behavior, stress-related behavior and substance-dependence (Mavridis, Psychiatriki. 2015 October-December; 25(4):282-94). The present invention has surprisingly found that a higher level of glutathione measured in the nucleus accumbens significantly correlates with improved motivational performance (FIG. 1) while stress as measured by cortisol levels is negatively correlated with levels of glutathione in the nucleus accumbens (FIG. 2).

The terms “food,” “beverage”, “food product”, “beverage product”, “food composition” and “beverage composition” mean a product or composition that is intended for ingestion by an individual such as a human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

The composition can be any kind of composition that is suitable for human and/or animal consumption. For example, the composition may be selected from the group consisting of: food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

As used herein, “complete nutrition” contains sufficient types and levels of macronutrients (protein, fats and carbohydrates) and micronutrients to be sufficient to be a sole source of nutrition for the individual to which the composition is administered. Individuals can receive 100% of their nutritional requirements from such complete nutritional compositions.

Administration of the compositions of the invention encompass “enteral administration” in all forms, although of oral administration is preferred.

Each of the compounds can be administered at the same time as the other compounds (i.e., as a single unit) or separated by a time interval (i.e., in separate units).

All modes of administration may be considered in combination with glutathione per se.

“Function derivatives” of compounds of the invention are derived from a similar compound by a chemical reaction. “Functional derivatives” can be formed from the same precursor compound and may be administered to increase glutathione levels in the brain.

### Embodiments

In several embodiments of the invention, a composition is provided for use in increasing glutathione levels in the brain wherein said composition comprises at least one compound selected from:

(i) compounds which are substrates or precursors of substrates of glutathione synthesis;

(ii) compounds which are targeting the regulation of anti-oxidant expression levels of glutathione by targeting Nrf2-dependent regulation and/or

(iii) compounds which further potentiate the antioxidant effect of glutathione.

In a preferred embodiment, the composition for use increases glutathione in the nucleus accumbens region of the brain to provide the benefits to the subject.

In several embodiments of the invention, a composition is provided for use in increasing glutathione levels in the brain wherein said composition comprises compounds of group (i) which are substrates or precursors of substrates of glutathione synthesis are selected from the group comprising: glycine, cysteine, glutamate, N-Acetylcysteine, taurine, and whey proteins, and/or their functional derivatives.

In several embodiments of the invention, a composition is provided for use in increasing glutathione levels in the brain wherein said composition comprises compounds of group (ii) which are targeting the regulation of anti-oxidant expression levels of glutathione by targeting Nrf2-dependent regulation are selected from the group comprising: sulforaphane, dimethylfumarate, curcumin, melatonin, and/or trehalose, and/or their functional derivatives.

In several embodiments of the invention, a composition is provided for use in increasing glutathione levels in the brain wherein said composition comprises compounds of group (iii) which further potentiate the antioxidant effect of glutathione are selected from the group comprising: puerarine, ergothioneine, l-carnitine, L-theanine, and/or glutamine and/or their functional derivatives.

In one embodiment of the invention, a composition is provided for use in increasing glutathione levels in the brain wherein said composition comprises glycine and N-acetylcysteine for use in increasing motivational performance or mental energy.

In one embodiment of the invention, a composition is provided for use in increasing glutathione levels in the brain wherein said composition comprises N-acetylcysteine, puerarine, sulforaphane for use in increasing motivational performance or mental energy.

In a further embodiment of the invention, a composition of the invention is administered with additional glutathione, preferably as S-acetyl glutathione for use in increasing motivational performance or mental energy.

In a preferred embodiment, a composition of the invention is administered orally.

In several embodiments of the invention, a composition of the invention is formulated as a food product, a food for special medical purposes (FSMP), a nutritional supplement, a ready to drink formula, a dairy-based drink, a low-volume liquid supplement, powder formats for liquid reconstitution, a meal replacement beverage, and combinations thereof.

In several embodiments of the invention, a composition of the invention is administered together with dietary recommendations for a diet rich in glutathione comprising foods selected from the group of: (a) cruciferous vegetables: broccoli, cauliflower, Brussels sprouts, and bok choy; (b) allium vegetables: garlic and onions; (c) eggs, nuts, legumes, fish, and chicken and/or (d) glutathione-rich herbs: milk thistle, flaxseed, guso seaweed.

In several embodiments of the invention, a composition of the invention is administered together with lifestyle recommendations to get at least 6 hours of sleep per night.

In several embodiments of the invention, a composition of the invention is used by healthy individuals in need of increasing motivational performance and/or mental energy.

In several embodiments of the invention, a composition of the invention is used by healthy individuals in need of increasing cognitive performance.

In several embodiments of the invention, a composition of the invention is used by healthy individuals in need of increasing motor performance.

In several embodiments of the invention, a composition of the invention is used by individuals suffering from low glutathione levels.

In several embodiments of the invention, a composition of the invention is used in a method of treatment to decrease performance anxiety.

In several embodiments of the invention, a composition of the invention is used in a method of treatment to decrease stress.

In several embodiments, a method of improving motivational performance and/or mental energy in a healthy subject is provided by administration of a glutathione enhancing composition according to the invention.

In several embodiments, a method of treating or preventing a condition associated with a reduced level of glutathione in the brain, is provided by administering to an individual in need thereof an effective amount of a combination of a composition of the invention.

### Glutathione

Glutathione (GSH) is the most abundant intracellular component of overall antioxidant defenses. GSH, a tripeptide, is synthesized from precursor amino-acids: glycine, cysteine and glutamate in two steps catalyzed by glutamate cysteine ligase (GCL, also known as gamma-glutamylcysteine synthetase, EC 6.3.2.2) and gamma-L-glutamyl-L-cysteine:glycine ligase (also known as glutathione synthetase, EC 6.3.2.3), and GSH synthesis occurs de novo in cells.

Glutathione is also known as Gamma-Glutamylcysteinylglycine, Gamma-L-Glutamyl-L-Cysteinylglycine, Gamma-L-Glutamyl-L-Cystéinylglycine, Glutathion, Glutatión, L-Gamma-Glutamyl-L-Cysteinyl-Glycine, L-Gamma-Glutamyl-L-Cystéinyl-Glycine, L-Glutathion, L-Glutathione, GSH, N—(N-L-gamma-Glutamyl-L-cysteinyl)glycine. It is typically administered as S-acetyl glutathione or reduced L-glutathione.

Glutathione-rich food include: cruciferous vegetables, for example, broccoli, cauliflower, Brussels sprouts, and bok choy; allium vegetables, for example, garlic and onions; eggs, nuts, legumes, lean protein, such as fish, and chicken as well as whey protein. Glutathione-rich herbs include: for example, milk thistle, flaxseed, guso seaweed. Compositions and methods of the invention can also be used in combination with dietary recommendations for a glutathione-rich food to complement the diet.

Lifestyle parameters may affect levels of glutathione in the brain. For example, glutathione is also negatively affected by insomnia. Therefore, compositions and methods of the invention would also include the recommendation to have sufficient sleep.

Psychogenic stress is defined as a state of imminent or perceived threat to homeostasis, where the brain and body invoke various physiological responses to adapt. Glutathione levels in the brain may be affected by such stress.

### Precursors of GSH

Precursors of GSH: glycine, cysteine or glutamate may be administered to increase the glutathione in the brain. Each of these precursors and their functional derivatives are described below:

### Glycine

Glycine or functional derivative thereof is selected from the group consisting of L-glycine, L-glycine ethyl ester, D-Allylglycine; N-[Bis(methylthio)methylene]glycine methyl ester; Boc-allyl-Gly-OH (dicyclohexylammonium) salt; Boc-D-Chg-OH; Boc-Chg-OH; (R)—N-Boc-(2′-chlorophenyl)glycine; Boc-L-cyclopropylglycine; Boc-L-cyclopropylglycine; (R)—N-Boc-4-fluorophenylglycine; Boc-D-propargylglycine; Boc-(S)-3-thienylglycine; Boc-(R)-3-thienylglycine; D-a-Cyclohexylglycine; L-a-Cyclopropylglycine; N-(2-fluorophenyl)-N-(methylsulfonyl)glycine; N-(4-fluorophenyl)-N-(methylsulfonyl)glycine; Fmoc-N-(2,4-dimethoxybenzyl)-Gly-OH; N-(2-Furoyl)glycine; L-a-Neopentylglycine; D-Propargylglycine; sarcosine; Z-a-Phosphonoglycine trimethyl ester, and mixtures thereof.

The glycine or functional derivative thereof can be administered in an amount of about 0.1-100 milligram (mg) of glycine or functional derivative thereof per kilogram (kg) of body weight of the subject.

In a particular non-limiting example, the daily doses for a 60 kg subject can be 6 to 6,000 mg/day for glycine or a functional derivative thereof.

### Cysteine and N-Acetylcysteine

Cysteine is a non-essential sulfur-containing amino acid important for protein synthesis, detoxification, and diverse metabolic functions. It is required for protein synthesis and for the synthesis of non-protein compounds including taurine, sulfate, coenzyme A, and GSH.

Cysteine itself is a powerful antioxidant and has the potential to trap ROS. Due to the fact that cysteine tends to be absorbed into cells where it cannot exhibit its antioxidant property, N-acetyl cysteine (NAC) is often used in supplement form instead for this purpose.

The N-acetylcysteine or functional derivative thereof can be administered in an amount of about 0.1-100 milligram (mg) of N-acetylcysteine (NAC) or functional derivative thereof per kilogram (kg) of body weight of the subject. In some embodiments, these amounts are provided at least partially by a dipeptide comprising both the N-acetylcysteine or functional derivative thereof and the glycine or functional derivative thereof.

In a particular non-limiting example, the daily doses for a 60 kg subject can be 6 to 6,000 mg/day of NAC or derivative thereof.

In one preferred embodiment, the glycine and the N-acetylcysteine may be formulated together in a particular ratio. In some embodiments, the formulation may comprise these components in the following exemplary ratios: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:300, 1:400, 1:500, 1:600, 1:750, 1:1000, and 1:10,000. In particular embodiments, the formulation may comprise these components in the following weight percentages (either the same for both glycine and the N-acetylcysteine or different weight percentages for each): 1%, 2%, 3%, 4%, 5%, 6%, 7%, 8%, 9%, 10%, 12%, 15%, 20%, 25%, 30%, 40%, 45%, 50%, 55%, 60%, 65%, 70%, 75%, 80%, 85%, 90%, 95%, 97%, or 99%, for example.

### Glutamate

Glutamate also known as L-glutamic acid supplies the amino group for the biosynthesis of other amino acids and is a substrate for glutamine and glutathione synthesis. It is the key neurotransmitter in the brain as well as an important energy source for certain tissues. The brain neurotransmitter glutamate is involved in a broad range of cognitive performance attributes of learning, behavior and memory. Glutamate acts as a co-substrate in the transamination and deamination of several amino acids. These reactions provide a carbon skeleton for glucogenesis or ATP generation.

The glutamate or functional derivative thereof can be administered in an amount of about 0.1-100 milligram (mg) of glutamate or functional derivative thereof per kilogram (kg) of body weight of the subject.

In a particular non-limiting example, the daily doses for a 60 kg subject can be 6 to 6,000 mg/day for glutamate or a functional derivative thereof.

(i) Other Compounds which are Substrates or Precursors of Substrates for Glutathione

Other substrates or precursors of substrates involved in the synthesis of glutathione are, for example, N-Acetylcysteine, taurine, whey proteins, and l-threonine which may be administered to increase glutathione in the brain.

### N-Acetylcysteine

The N-acetylcysteine or functional derivative thereof can be administered in an amount of about 0.1-100 milligram (mg) of N-acetylcysteine (NAC) or functional derivative thereof per kilogram (kg) of body weight of the subject. In some embodiments, these amounts are provided at least partially by a dipeptide comprising both the N-acetylcysteine or functional derivative thereof and the glycine or functional derivative thereof.

In a particular non-limiting example, the daily doses for a 60 kg subject can be 6 to 6,000 mg/day of NAC or derivative thereof.

### Taurine

Taurine also known as 2-aminoethanesulfonic acid is an organic acid that occurs naturally in food, especially in shellfish (eg, scallops, mussels, clams) and in the dark meat of turkey and chicken, as well as in other meats and eggs.

In a particular non-limiting example, the daily doses for a 60 kg subject up to 3000 mg/day of taurine or a functional derivative thereof.

### Whey Proteins

Whey proteins and whey protein isolates contain high amounts of amino acids such as glycine, cysteine and leucine, especially in isolates enriched for alpha-lac.

In a particular non-limiting example, the daily dose of whey protein is 0.8 to 2.5 g/kg body weight of whey proteins per day.

### (ii) Compounds Targeting Regulation of Antioxidant Expression Via Nrf2

Compounds targeting regulation of antioxidant expression via Nrf2 may be administered to increase the glutathione in the brain. For example, sulforaphane, dimethylfumarate, curcumin, melatonin, and trehalose.

### Sulforaphane

Sulforaphane exists in food in its food-bound form known as Glucoraphanin, a glycoside (bound to a sugar) or sulforaphane that is commonly seen as a prodrug or storage form of Sulforaphane.

In a particular non-limiting example, the daily dose of sulforaphane or a functional derivative thereof may be administered at 7 to 57 mg/day.

### Dimethylfumarate

Dimethylfumarate and its active metabolite, monomethyl fumarate (MMF), have been shown to activate the nuclear factor erythroid-derived 2-like 2 (Nrf2) pathway

In a particular non-limiting example, the daily dose of dimethylfumarate or a functional derivative thereof, such as monomethylfumarate, may be administered at 120-240 mg/day for up to one week and such administration should be monitored by a physician.

### Curcumin

Curcumin is the key active ingredient in the yellow-colored powder ground from the root of the turmeric plant.

In a particular non-limiting example, the daily dosing of curcumin or a functional derivative thereof may be administered at 500 to 1500 mg/day.

### Melatonin

Melatonin also known as 5-Methoxy-N-Acetyltryptamine is a hormone found naturally in the body but can also be made synthetically.

In a particular non-limiting example, the daily dosing of melatonin or a functional derivative thereof may be administered at 0.5 to 12 mg/day.

### Trehalose

Trehalose is a naturally-occurring disacharide with antioxidant properties, which has been shown to regulate the Keap1-Nrf2 pathway. It may need to be administered as an injection or topically as trehalose is poorly absorbed from the intestine.

In a particular non-limiting example, the daily dosing of trehalose or a functional derivative thereof may be administered at up to 50 g/day.

(iii) Compounds which are Antioxidants Influencing Glutathione

In several embodiments, compounds which are antioxidants influencing glutathione may be administered to increase the glutathione in the brain. For example, puerarine, ergothioneine, l-carnitine, and glutamine.

### Puerarine

Puerarine is an isoflavone and the major bioactive ingredient isolated from the root of the Pueraria lobate also known as Kudzu plant.

In a particular non-limiting example, the daily dosing of puerarine or a functional derivative thereof may be administered at 1.5 to 3.0 g/day from a root extract.

### Ergothioneine

Ergothioneine is also known as ergothionine, 1-carboxy-2-[2-mercaptoimidazole-4-(or 5)-yl]ethyl]-trimethyl-ammonium hydroxide, 2-Mercaptohistidine Trimethylbetaine, or l-ergothioneine. It is an amino acid that is found mainly in mushrooms, but also in king crab, meat from animals that have grazed on grasses containing ergothioneine, and other foods.

In a particular non-limiting example, the daily dosing of ergothioneine or a functional derivative thereof may be administered at 2 to 25 mg/day.

### L-Carnitine

L-carnitine is an amino acid that is produced in the body but can be taken as a supplement. The body can convert L-carnitine to other amino acids such as acetyl-L-carnitine and propionyl-L-carnitine.

In a particular non-limiting example, the daily dosing of l-carnitine or a functional derivative thereof may be administered at 900 mg to 4000 mg/day.

### L-Theanine

L-theanine is synthesized from glutamic acid and ethylamide and found in foods such as green tea. It is not an antioxidant itself but promotes glutathione.

In a particular non-limiting example, the daily dosing of l-theanine or a functional derivative thereof may be administered at 50 mg to 200 mg/day.

### Glutamine

The glutamine or functional derivative thereof can be administered in an amount of about 0.1-100 milligram (mg) of glutamine or functional derivative thereof per kilogram (kg) of body weight of the subject.

In a particular non-limiting example, the daily doses for a 60 kg subject can be 6 to 6,000 mg/day for glutamine or a functional derivative thereof.

### Composition Formulations

### Food Compositions

In one embodiment, the compositions are food compositions, including human and pet food compositions. In several embodiments, the food composition is a product with at least one nutrient for improving motivation performance or mental energy.

For pet food compositions, they may supply the necessary dietary requirements for an animal, animal treats (e.g., biscuits), or dietary supplements. The compositions may be a dry composition (e.g., kibble), semi-moist composition, wet composition, or any mixture thereof. In another embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement is to be administered to the animal in small amounts, or in the alternative, can be diluted before administration to an animal. The dietary supplement may require admixing, or can be admixed with water or other diluent prior to administration to the animal.

### Beverage Compositions

In one embodiment, the compositions are beverage compositions. Such beverage compostions are meant to be consumed by a human or animal. In several embodiments, the beverage is a milk based beverage; a performance nutrition product, a medical nutrition product; a milk product, e.g. a milk drink, a product with at least one nutrient for improving motivation performance or mental energy.

### Dairy Product

In one embodiment, the composition can be formulated as a “dairy product” together with milk proteins, e.g., milk protein concentrate or milk protein isolate; caseinates or casein, e.g., micellar casein concentrate or micellar casein isolate; or whey protein, e.g., whey protein concentrate or whey protein isolate. Additionally or alternatively, at least a portion of the protein can be plant protein such as one or more of soy protein, pea protein or canola protein.

### Nutritional Supplement

In one embodiment, the composition of the invention can be formulated as a “nutritional supplement” together with glutathione enhancing compounds of the invention. The compounds of the invention can be used alone or in combination with appropriate additives to make tablets, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

### Administration

The composition of the invention can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the condition remain. In specific embodiments, the administration occurs until a detectable improvement of at least one symptom occurs and, in further cases, continues to remain ameliorated.

The ideal duration of the administration of the composition can be determined by those of skill in the art.

The compositions disclosed herein may be administered to the subject orally or parenterally, preferably orally. Non-limiting examples of parenteral administration include intravenously, intramuscularly, intraperitoneally, subcutaneously, intraarticularly, intrasynovially, intraocularly, intrathecally, topically, and inhalation. As such, non-limiting examples of the form of the composition include natural foods, processed foods, natural juices, concentrates and extracts, injectable solutions, microcapsules, nano-capsules, liposomes, plasters, inhalation forms, nose sprays, nosedrops, eyedrops, sublingual tablets, and sustained-release preparations.

The active agent may be systemic after administration or may be localized by the use of regional administration, intramural administration, or use of an implant that acts to retain the active dose at the site of implantation.

The compounds can be formulated into preparations for injections by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in an aerosol formulation to be administered by inhalation. For example, the compounds can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds can be administered rectally by a suppository. The suppository can include a vehicle such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition. Similarly, unit dosage forms for injection or intravenous administration may comprise the compounds in a composition as a solution in sterile water, normal saline or another pharmaceutically acceptable carrier, wherein each dosage unit, for example, mL or L, contains a predetermined amount of the composition containing one or more of the compounds.

### EXAMPLES

### Example 1: Nucleus Accumbens Glutathione Predicts Human Motivational Performance

The following abbreviations refer respectively to the definitions below: GSH (Glutathione); TAU (Taurine); HA (High Anxious); LA (Low Anxious); NAc (Nucleus Accumbens); VS (ventral striatum); 1H MRS (proton magnetic resonance spectroscopy); MID (monetary incentive delay); Cort (cortisol)

The role of glutathione concentrations in human motivated effort was examined. Following basal personality characterization, baseline metabolite concentrations were measured using proton resonance spectroscopy in a 7 T/68 cm MR scanner (Magnetom, Siemens Medical Solutions, Erlangen, Germany) as described below. Following acquisition, participants were invited to participate in an MID task to measure their motivational performance. Cortisol samples were collected throughout the experiment to determine participant stress levels.

### Participants

Forty-three healthy male individuals from the EPFL and UNIL campus in Lausanne (Switzerland) were recruited according to the following criteria: male, 20-30 years old, right-handed, no regular drug or medication intake, non-smoker, no history of psychiatric or neurological illness, and no metallic implants. Out of these 43 participants, data for 16 participants was not collected due to the following reasons: 4 participants dropped out voluntarily on the scanning day, 4 participant reported metal implants on the scanning day, 2 participants were not suited for the scanner environment due to anthropometric limitations which could not be foreseen, 4 participants moved during neuroimaging which resulted in unsuccessful shimming and for 2 participants a hardware connection cable was unplugged leading to the acquisition of faulty data. Therefore, data from 27 participants were used in the study.

Experiments were performed between noon and 6 μm. Participants were instructed not to eat or drink any caffeinated drinks one hour before the experiment, and not to arrive to the laboratory hungry or thirsty. They were instructed not to take any medication and to avoid physical effort within 24 hours before the experiment. Informed written consent was obtained from all participants. The study was approved by the Cantonal Ethics Committee of Vaud, Switzerland.

### Personality Questionnaires and Anthropometric Characteristics

We have recently shown the effect of trait anxiety and effort allocation in the MID task applied in this study (Berchio et al., Translational Psychiatry, 9, 2019). To control for this aspect of task performance, we assessed state and trait anxiety with the State-Trait Anxiety Inventory (STAI) (Calixto et al., International Journal of Nanomedicine, 9(1), 3719-3735, 2014). Due to the previously reported role of social dominance on NAc mediated competitive behavior in rats (Hollis et al., Proceedings of the National Academy of Sciences, 112(50), 15486-15491, 2015), we controlled for this aspect with the Personality Research Form (PRF) dominance scale which measures dominance motivation in humans (Jackson, Personality research form manual. Research Psychologists Press, 1974). Likewise, we controlled for competitiveness with the revised competitiveness index capturing interpersonal competitiveness in everyday contexts (Houston et al. Psychological Reports, 90(1), 31-34, 2002), self-perceived social rank with the social comparison scale (Gilbert et al., New Ideas in Psychology, 13(2), 149-165, 1995), participants' physical fatigue (state and trait) and physical activity with the Mental and Physical State Energy and Fatigue Scales (SEF) (Loy et al., Physiology and Behavior, 153, 7-18, 2016). In addition to the above scales, we measured age, height, weight, computed the BMI and elicited individuals' maximal voluntary contraction (MVC), the latter procedure is described in the Methods below. Both before and at various time points during the experiment, saliva samples were collected using salivettes (Sarstedt). Cortisol concentrations were measured using a cortisol ELISA kit (Enzo Life Sciences) following the manufacturer's protocol.

### Proton Magnetic Resonance Spectroscopy (¹H MRS) Acquisition and Data Processing

The MR measurements were performed on a 7 T/68 cm MR scanner (Magnetom, Siemens Medical Solutions, Erlangen, Germany) with a single-channel quadrature transmit and a 32-channel receive coil (Nova Medical Inc., MA, USA). MR images were acquired with a magnetization-prepared rapid gradient-echo (MP2RAGE) sequence for MRS voxel positioning with the following parameters: repetition time (TR)=5500 ms, echo time (TE)=1.87 ms, inversion time (TI)₁=750 ms, TI₂=2350 ms, α₁=4°, α₂=5°, 1×1×1 mm resolution, matrix size=210×210×160] (Marques et al., NeuroImage, 49(2), 1271-1281, 2010).

The NAc voxel was defined by the third ventricle medially, the subcallosal area inferiorly, and the body of the caudate nucleus and the putamen laterally and superiorly, in line with definitions of NAc anatomy identifiable on MRIs (Neto et al., Neuromodulation, 11(1), 13-22, 2008).

Magnetic field inhomogeneities within the VOI were minimized using 1^{st}- and 2^{nd}-order shims with the fast, automatic shim technique using echo-planar signal readout for mapping along projections FAST(EST)MAP sequence (Gruetter, Magnetic Resonance in Medicine, 29(6), 804-811, 1993; Gruetter et al., Magnetic Resonance in Medicine, 43(2), 319-323, 2000).

¹H MR spectra were acquired in the NAc with the semi-adiabatic spin-echo full-intensity acquired localized (semi-adiabatic SPECIAL) sequence (Xin et al., Magnetic Resonance in Medicine, 69(4), 931-936, 2013) in the NAc (VOI=14×10×13 mm³, TR/TE=6500/16 ms, bandwidth=4000 Hz, vector size=2048 pts, average of 256) and OL (VOI=25×20×20 mm³, TR/TE=8000/16 ms, bandwidth=4000 Hz, vector size=2048 pts, average of 64) including 6 outer volume suppression bands and water suppression using the variable pulse power and optimized relaxation delays (VAPOR) sequence (Tkáć et al., Applied Magnetic Resonance, 29(1), 139-157, 2005). The unsuppressed water signal was acquired and used as an internal reference for the metabolite quantification and eddy current correction.

Localized single-voxel ¹H MR spectra from the left accumbens were obtained from twenty-seven and seventeen participants. ¹H MRS in the bilateral occipital lobe was performed as the experimental control on a different day in the participants that were successfully recruited to return to the laboratory (n=17).

MR images were segmented and grey matter (GM), white matter (WM) and cerebrospinal fluid (CSF) percentage inside the MRS voxel were evaluated (Van Leemput et al., IEEE Transactions on Medical Imaging, 18(10), 885-896, 1999) and used to calculate water concentration assuming water concentrations of 43300 mM in GM, 35880 mM in WM, and 55556 mM in CSF (Provencher, LCModel & LCMgui user's manual. LCModel Version, 6-2, 2014). Metabolite concentrations were then partial-volume corrected for the CSF fraction.

¹H MR spectra were frequency corrected, summed and then analyzed with LCModel with a basis set including simulated metabolite spectra and an experimentally measured macromolecule baseline (Govindaraju et al, NMR in Biomedicine, 13(3), 129-153, 2000; Provencher, Magnetic Resonance in Medicine, 30(6), 672-679, 1993; Schaller et al., Magnetic Resonance in Medicine, 72(4), 934-940, 2014).

### Modified Monetary Incentive Delay (MID) Task

Participants performed a modified version of the monetary incentive delay (MID) task (B Knutson et al., J Neurosci, 21(16), 2001). Our modified MID version relied on exerting force on a hand grip or dynamometer (TSD121B-MRI, Biopac) at a threshold corresponding to 50% of the participant's maximum voluntary contraction (MVC). To set individuals' task threshold, each participant was instructed to exert as much force as possible on the dynamometer for 1 sec, repeated 3 times interspersed by breaks of 3 min each to allow for recovery (Voor et al., Journal of Motor Behavior, 1(3), 210-219, 1969). The highest out of the 3 values was used to calibrate the threshold for the handgrip force required to be exerted in the modified MID task. Force (in kilogram, kg) was recorded in Acknowledge 4.3 (BIOPAC Systems, United States). Visual signal inspection confirmed the absence of artefacts.

Participants were comfortably seated in front of a computer screen at 90 cm distance and were instructed to keep the same right upper limb position (i.e., upper arm and forearm at 90° angle and hand extended) whenever using the dynamometer. The signal recorded with the dynamometer, linearly proportional to the exerted force (in kg), was fed back to the stimuli presentation PC (running the MID task in E-Prime) in real-time. The task was run in 2 blocks with a 3 min break between the 2 blocks. Each block contained 2 sessions and each session had twenty trials: 5 incentivized trials of each of the different incentives (0.2, 0.5, 1) in a random order, and 5 non-incentivized rest trials interspersed within every 3 incentivized trials. Our modified MID task had eighty total trials. To earn the displayed incentives, the participant's 50% MVC threshold had to be reached within 2 sec and the force at or above the threshold maintained for another 3 sec. Performance was guided by visual cues on the screen that were adapting to participants' performance in real-time.

During the task, trials started with a fixation cross (varying between 1-4 sec), followed by an anticipatory signal (3 sec) indicating the trial's incentive. To earn the monetary incentives, participants were instructed to exert force on the dynamometer. The beginning of the force exertion period was signaled by the appearance of a red circle around the fixation cross. If the established threshold (i.e. 50% of the participant's MVC force—0.5 kg) was reached within 2 sec, the red circle was replaced by a green circle. The green circle also indicated that participants had to maintain the contraction force level above the threshold for 3 more seconds. If participants did not reach the threshold in the initial 2 sec or if the force level fell below the maintenance threshold during the 3 sec maintenance period, the trial was failed, and visualized by a red cross occurring on the screen. If the force was maintained for the required 3 sec, a green tick indicated successful task performance during a single trial. Total trial duration was fixed to 10 sec. Participants performed 1 pre-training session of 20 trials (as described above) during which no incentives could be earned.

Success was computed in % of successful trials out of total trials, and for each of the four sessions (i.e. Success_{Total}, Successs_{Session 1}, Success_{Session 2}, Success_{Session 3}, Success_{Session 4}) and for each of the three incentives (i.e. CHF 0.2, 0.5 and 1).

At the end of the experiment, participants were asked to estimate the threshold at which their force was successful to activate the green circle during the experiments, on a scale from 10% to 120% of their MVC, with steps of 10%.

### Statistical Analyses

Data was processed and analyzed in IBM SPSS Statistics 20, MATLAB R2017a, and GraphPad Prism 7. Kolmogorov-Smirnov tested for normality distributions in the data.

Associations were quantified with Pearson's correlation coefficient for normally distributed variable pairs. Associations including not normally distributed variables were quantified with Spearman rank correlation coefficients. Correlation coefficients were compared according to Zou's confidence intervals.

Due to lack of previously published data involving the variables measured in this study, all statistical tests were run two-sided. Statistical testing was performed with an alpha level of 0.05.

FIG. 1 shows that GSH concentrations measured in the nucleus accumbens by 1H MRS significantly correlates with total performance in a subsequent monetized hand grip effort task.

FIG. 2 shows that GSH concentrations measured in the nucleus accumbens by 1H MRS are negatively correlated with changes in cortisol levels sampled during a hand grip effort task.

### Example 2: Glutathione and Taurine Levels Measured During Anxiety

The role of trait anxiety in basal GSH and TAU concentrations was examined. Mice were characterized for their natural trait anxiety as described below in the methods section. Metabolites were then measured using ¹H MRS. The following abbreviations refer respectively to the definitions below: GSH (Glutathione); TAU (Taurine); HA (High Anxious); LA (Low Anxious); NAc (Nucleus Accumbens); VS (ventral striatum); 1H MRS (proton magnetic resonance spectroscopy); MID (monetary incentive delay); Cort (cortisol)

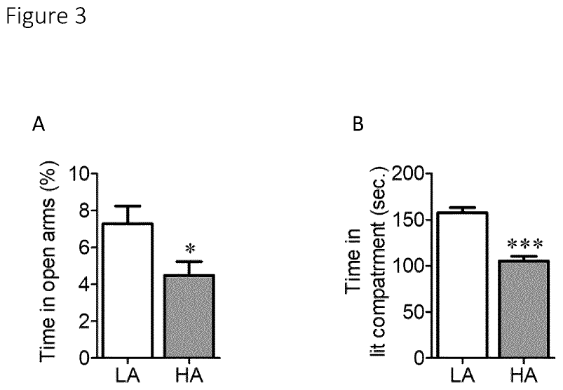

FIG. 3 shows how inbred mice can be classified as either high or low anxious according to their behavior in tests of anxiety-like behavior. (A) High anxious mice spend significantly less time in the open arms of an elevated plus maze. (B) High anxious mice spend significantly less time in the anxiogenic lit compartment of a light-dark box.

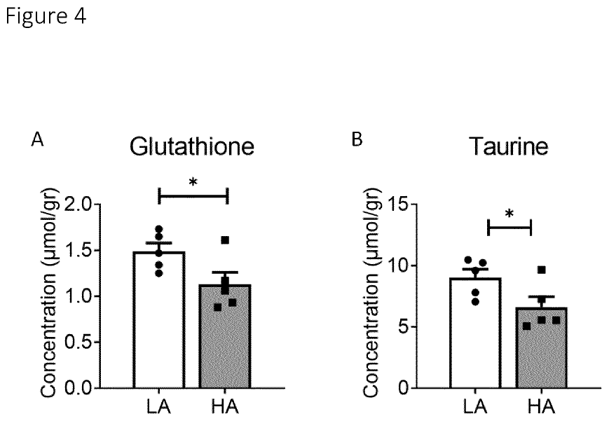

FIG. 4 shows how mice characterized for their natural trait anxiety in an elevated plus maze exhibit significant differences in GSH concentrations in the nucleus accumbens under basal conditions.

### Methods

All experiments were performed with the approval of the Cantonal Veterinary Authorities (Vaud, Switzerland) and carried out in accordance with the European Communities Council Directive of 24 Nov. 1986 (86/609EEC). All experiments were performed on C57B16/J mice obtained from Charles River Laboratories. After arrival, animals were housed four per cage and allowed to acclimate to the vivarium for one week. All animals were subsequently handled for 1 min per day for a minimum of 3 days. Animals were weighted upon arrival as well as weekly to ensure good health. Mice were maintained under standard housing conditions on corn cob litter in a temperature- (23±1_C) and humidity (40%)-controlled animal room with a 12-h light/dark cycle (0700-1900 hr), with ad libitum access to food and water. All tests were conducted during the light period.

### General Experimental Design

One week after their arrival, mice were tested at 7-weeks-old in an elevated plus maze and light-dark box for their basal anxiety between 08 h00 and 09 h00. Thirteen week-old mice were then exposed to metabolite measurements by spectroscopy.

### Elevated Plus Maze Test

The apparatus was made from black PVC with a white floor. The apparatus consisted of a central platform (5 3 5 cm) elevated from the ground (65 cm) from which two opposing open (30 3 5 cm) and two opposing (30 3 5 3 14 cm) close arms emanated. Light conditions were maintained at 14-15 lx in the open arms, and 3-4 lx in the closed arms. At the start of the test, animals were placed at the end of the closed arms faced to the wall, after which the animals were allowed to freely explore the apparatus for 5 min. Mice were tracked (Ethovision 11.0 XT, Noldus, Information Technology) to measure the time spent in the open-arms, closed arms and, the risk zones (edge of the open arms).

### Light-Dark BOX

Anxiety-like behaviors were also evaluated in a light-dark box, as previously described (Bisaz and Sandi 2010). A 27×27×26-cm lit (room light 45-50 lx) white compartment with open top was connected through an opening entrance (5×5 cm) to a 27×27×26-cm black box compartment covered with a lid. Each subject was placed in the center of the dark compartment and total distance traveled, frequency of entries, and percent time in the light compartment were recorded using video tracking for 5 min (EthoVision 3.0, Noldus). Differences in the number of entries and the time spent in the light compartment were considered as indicators of anxiety-related behaviors. Between sessions, both compartments were cleaned with 5% ethanol/water.

### ¹H-NMR Spectroscopy

All spectroscopic measurements were performed on animals after at least one week of acclimation upon arrival. Animals were anesthetized with 3% isoflurane for induction and fixed on an in-house-built holder with biting piece and ear bars. Animal physiology was maintained stable under 1.3%-1.5% isoflurane in a 1:1 air/oxygen mixture and was monitored for breathing (small animal monitor system: SA Instruments Inc., New York, N.Y., USA) and rectal temperature (circulating water bath). Body temperature was maintained at 36.5±0.4_C and breathing rate ranged between 70-100 rpm. Maximal time under anesthesia was 2 h for each animal. Mice were scanned with a horizontal 14.1 T/26 cm Varian magnet (Agilent Inc., USA) and a homemade 1H surface coil in quadrature consisting of two geometrically decoupled loops used as radio frequency (RF) transceiver. Coronal T2-weighted fast spin echo (FSE) images were obtained (15 3 0.4 mm slices, TEeff/TR=50/2000 ms, averages=2) for volumes of interest (VOIs) localization. VOIs included medial prefrontal cortex (mPFC) (1.7×1.4×1.2 mm3) and bilateral nuclei accumbens (NAc) (1.4×4.1×1 mm3). Field homogeneity was adjusted using first- and second order shims obtained using the FASTMAP protocol [49] to reach a water linewidth under 20 Hz. The VAPOR module was used for water suppression and outer volume suppression was performed to avoid spectra artifacts. Spectra were obtained using the spin echo full intensity acquired localized (SPECIAL) sequence on the target VOIs (TE/TR=2.8/4000 ms) [50] in blocks of 16 averages. Scan time was adjusted in order to obtain a satisfactory SNR (i.e. >10) and was in average around 20 min for NAc and 25 min for mPFC. Spectra were frequency corrected using the Creatine (Cr) frequency peak at 3.03 ppm as reference and blocks were summed for quantification. The LCModel [51] method, which is based on a linear combination of metabolite resonance peaks, was used to quantify the spectra in the frequency domain. For each animal, nineteen individual metabolites together with the macromolecule signals were quantified [alanine (Ala), ascorbate (Asc), aspartate (Asp), gamma-amino butyric acid (GABA), N-acetylaspartate (NAA), N-acetyl-aspartate glutamate (NAAG), glutathione (GSH), Cr, phosphocreatine (PCr), glutamate (Glu), glutamine (Gln), lactate (Lac), taurine (Tau), myoinositol (Ins), glycine (Gly), phosphorylcholine (PCho), glycerophosphocholine (GPC), glucose (Glc), phosphorylethanolamine (PE)]. An unsuppressed water spectrum was acquired before each MRS scan and was used as a reference for metabolite absolute concentration determination assuming 80% water content in the brain. Fitting reliability was determined using the Cram_er-Rao lower bound errors (CRLB). A threshold of CRLB % 20% was chosen for high concentrated metabolites and CRLB % 50% for low concentration metabolites. Similarity in macromolecule content was used to control for reliable metabolite quantification between groups as these molecules were assumed to be constant.

### Statistical Analyses

All values are given as mean±s.e.m. Results were analyzed for differences between trait anxiety groups by an unpaired t-test. Results obtained in the spectroscopy scans under basal conditions were analyzed by a one-way analysis of variance (ANOVA), with anxiety trait as a fixed factor. Analyses were followed by the Bonferroni post hoc test when appropriate. All statistical tests were performed with GraphPad Prism (GraphPad software, San Diego, Calif., USA) using a critical probability of p<0.05.

### Example 3—Cytoplasmic Reactive Oxygen Species (ROS) Measurements

Cytoplasmic ROS was measured in rat primary astrocytes in culture by CellROX® Deep Red reagent, a novel cell-permeant dye with absorption/emission maxima of ˜644/665 nm. CellROX® Deep Red reagent while in a reduced state is non-fluorescent and becomes fluorescent upon oxidation by reactive oxygen species with emission maxima ˜665. CellMask™-green stains plasma membrane and DAPI stains nuclei. We used the green channel and the dapi to segment the image in cytoplasmic and nuclear areas and measure the CellROX deep red signal in these areas. As CellROX® Deep Red is a better read out for cytoplasmic ROS, we measure the average intensity in the cytoplasm and normalized to the average cytoplasmic area. Cell count using DAPI was performed to establish toxicity or cell detachment for each condition. Images were taken to cover all the area of the well where cells were seeded and at least 4 technical replicates were done for each condition and for each biological replicate.

Measurements were performed at 48 hrs after treatment at baseline and after oxidative stress where oxidative stress was triggered by 250 μM tBHP for 1 hour.

Results were compared to the control condition at baseline for each biological replicate and for each condition.

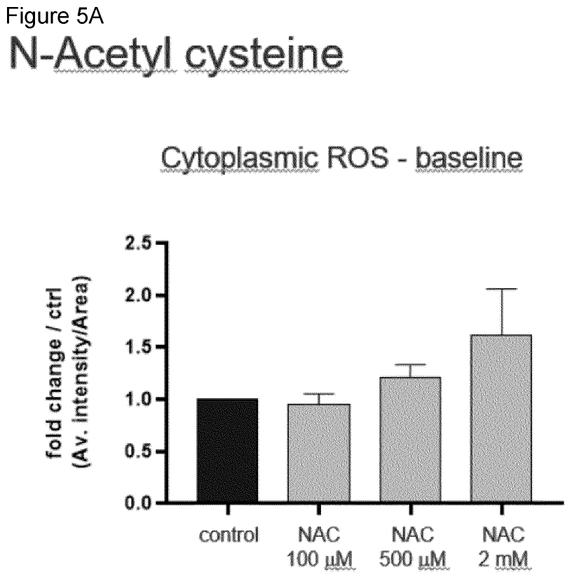

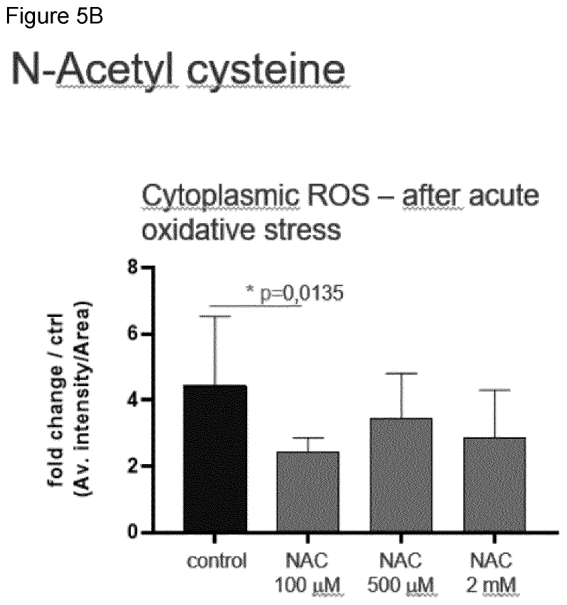

FIG. 5A shows the result of N-Acetylcysteine at baseline and FIG. 5B after oxidative stress.

N-Acetylcysteine decreases reactive oxygen species after oxidative stress.

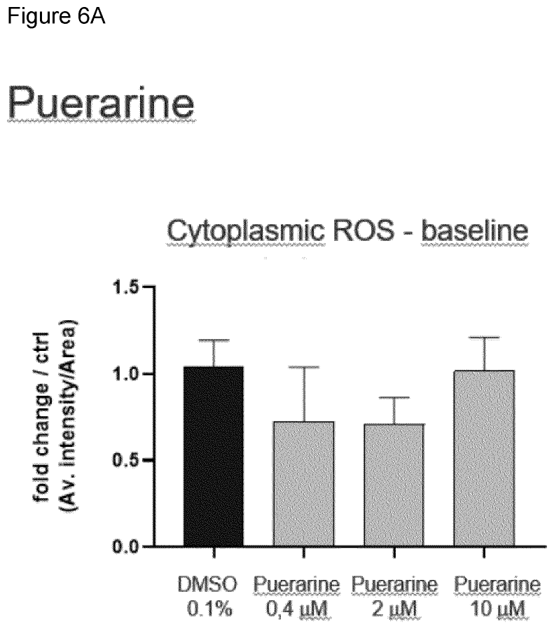

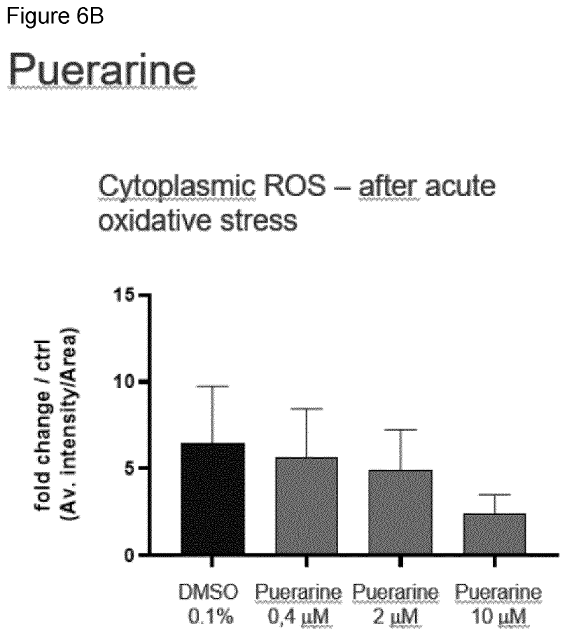

FIG. 6A shows the result of Puerarine at baseline and FIG. 6B after oxidative stress.

Puerarine decreases reactive oxygen species after oxidative stress in a dose dependent manner.

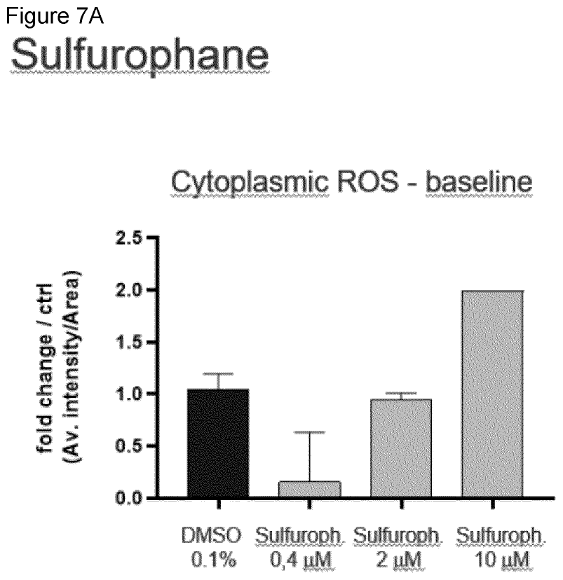

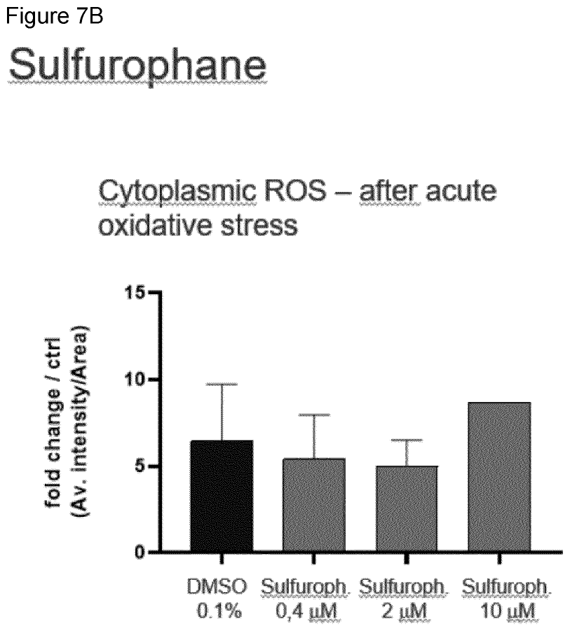

FIG. 7A shows the result of Sulfurophane at baseline and FIG. 7B after oxidative stress.

Sulfurophane decreases reactive oxygen species after oxidative stress.

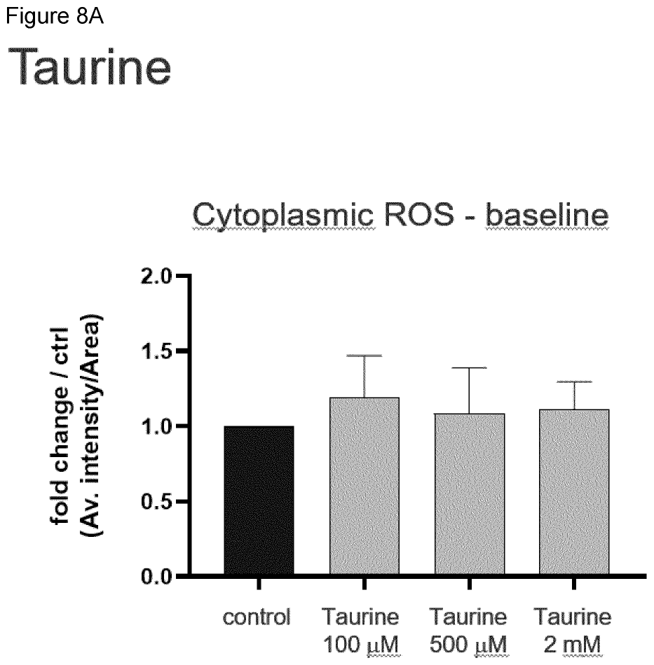

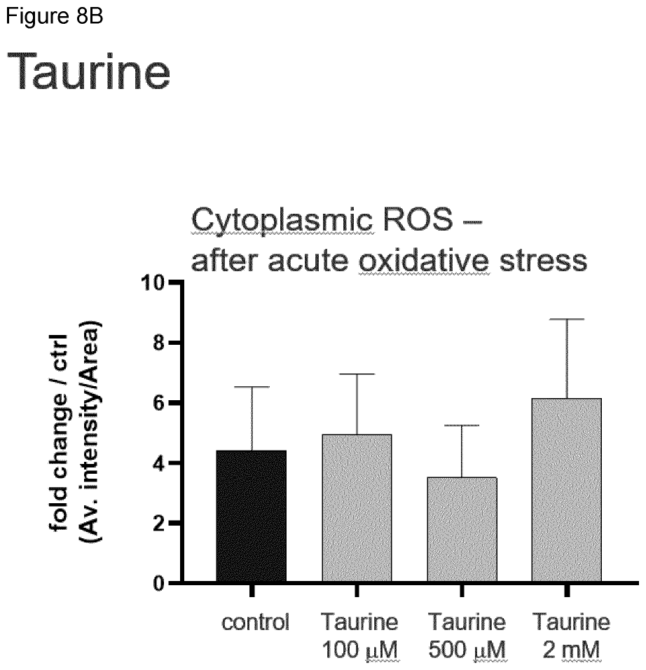

FIG. 8A shows the result of Taurine at baseline and FIG. 8B after oxidative stress.

Taurine decreases reactive oxygen species after oxidative stress.

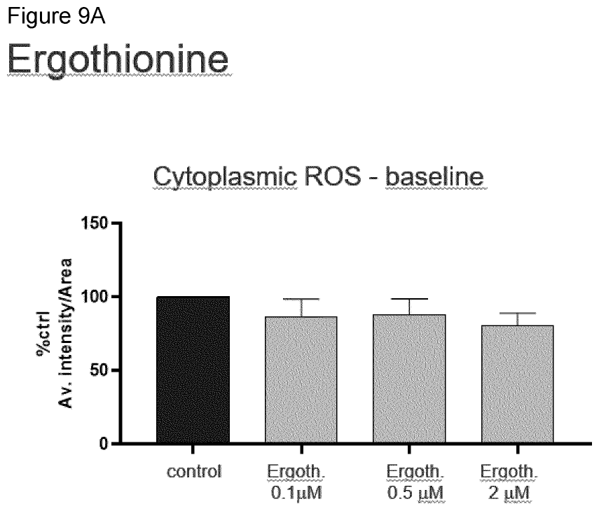

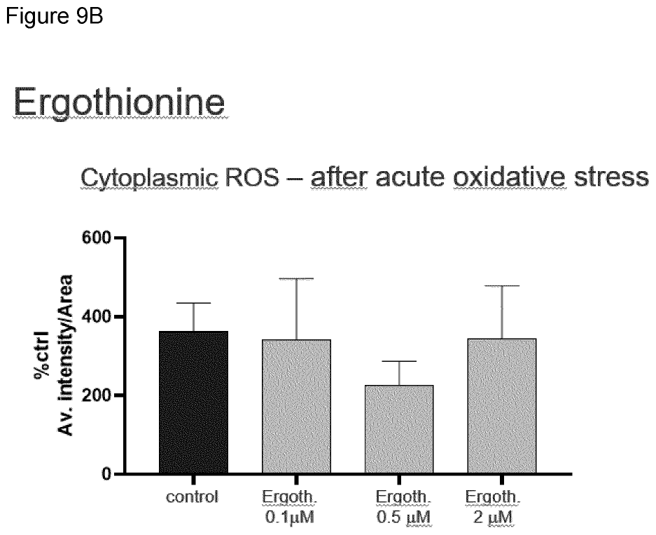

FIG. 9A shows the result of Ergothioneine at baseline and FIG. 9B after oxidative stress.

Ergothioneine decreases reactive oxygen species after oxidative stress.

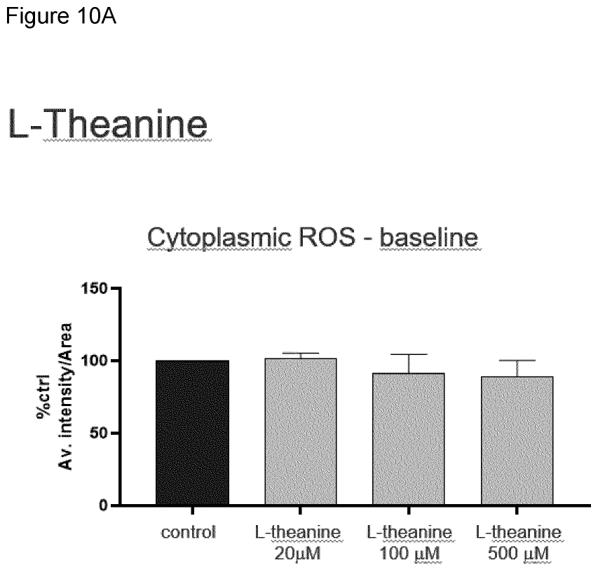

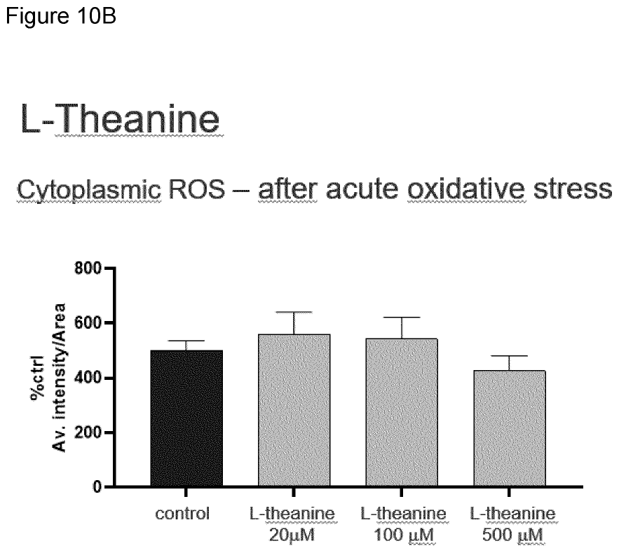

FIG. 10A shows the result of L-theanine at baseline and FIG. 10B after oxidative stress.

L-theanine decreases reactive oxygen species after oxidative stress.

### Example 4—Measurement of Nuclear Nrf2 in Astrocytes

Nuclear Nrf2 was measured in rat primary astrocytes in culture. The cells were stained with Nrf2 (Abcam ab89443 1:500), Tubulin (Abcam ab 89984 1:1000) antibodies and DAPI as nuclear counterstaining. We used the tubulin channel and the DAPI to segment the image in cytoplasmic and nuclear areas and measure the Nrf2 signal in these areas. We measure the average intensity in the nucleus and normalized to the average nuclear area. Cell count using DAPI was performed to establish toxicity or cell detachment for each condition. Images were taken to cover all the area of the well where cells were seeded and at least 6 technical replicates were done for each condition and for each biological replicate.

Measurements were performed 48 hrs after treatment with each of the compounds: N-Acetylcysteine dissolved in water, while Puerarine, Sulfurophane, Taurine at different doses were dissolve in a 0.1% DSMO final solution. They were compared to the control in each condition which was water or 0.1% DSMO alone. Results were compared to the control condition for each biological replicate and for each condition. Statistics were done using Kruskal-Wallis Multiple Comparison test.

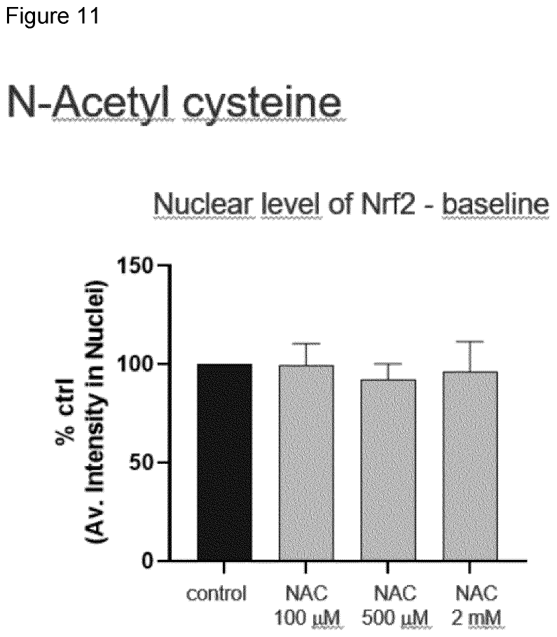

FIG. 11 shows that N-Acetylcysteine does not activate Nrf2 hence having no effect on glutathione genes.

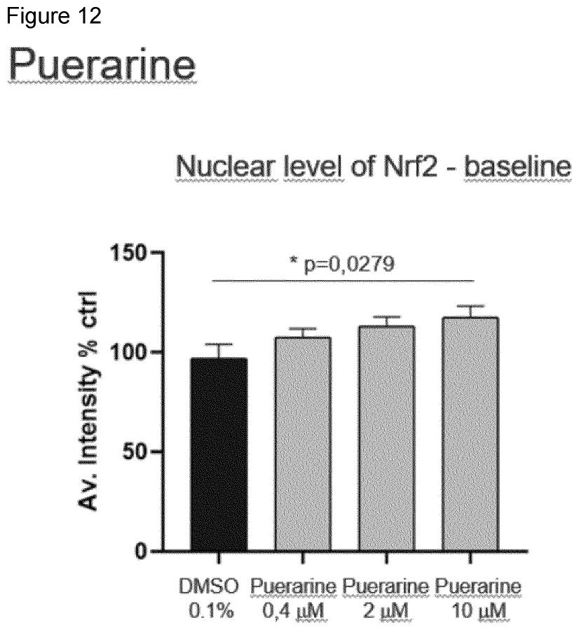

FIG. 12 shows that Puerarine significantly activates Nrf2 at the highest dose of 10 μm with up to a 20% increase Nrf2 levels in the cell nucleus.

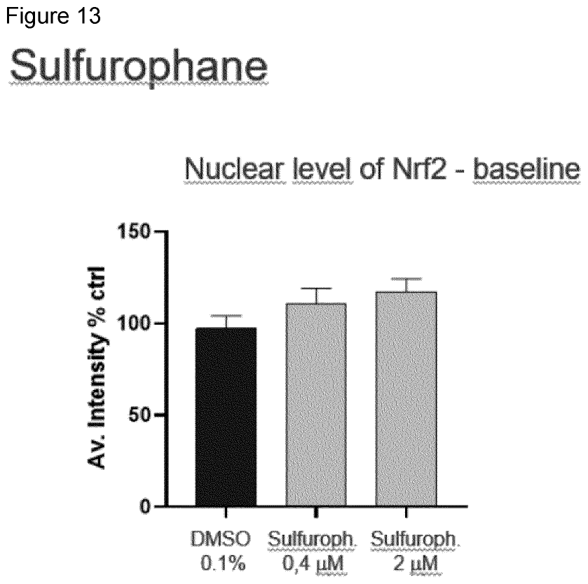

FIG. 13 shows that Sulfurophane significantly activates Nrf2 at 2 μM with up to a 20% increase Nrf2 levels in the cell nucleus.

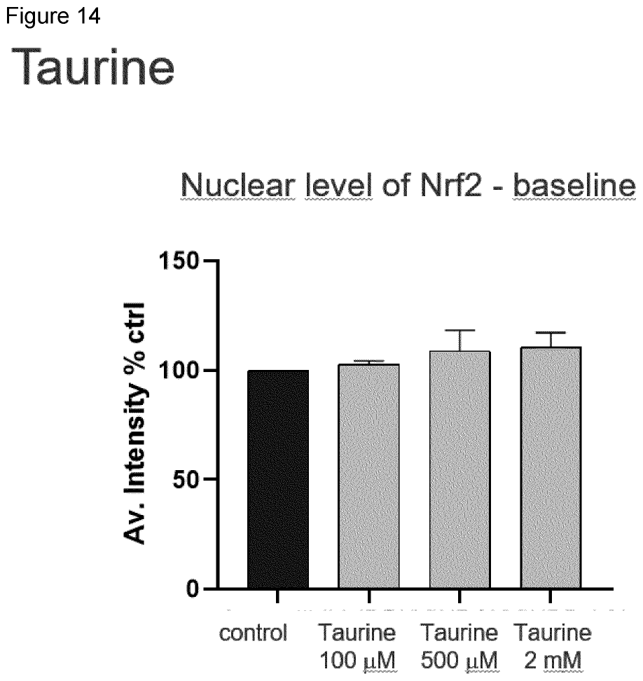

FIG. 14 shows that Taurine does not activate Nrf2.

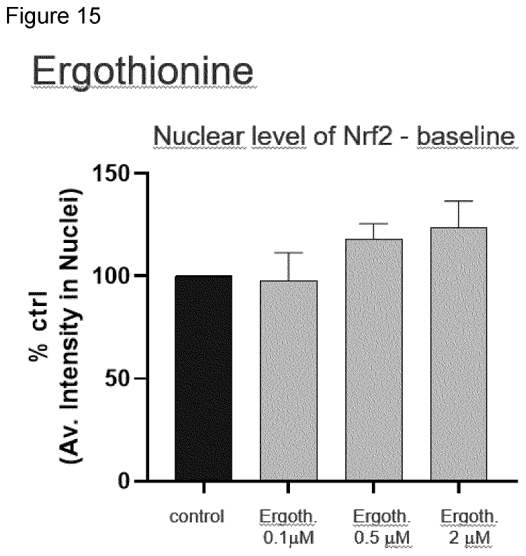

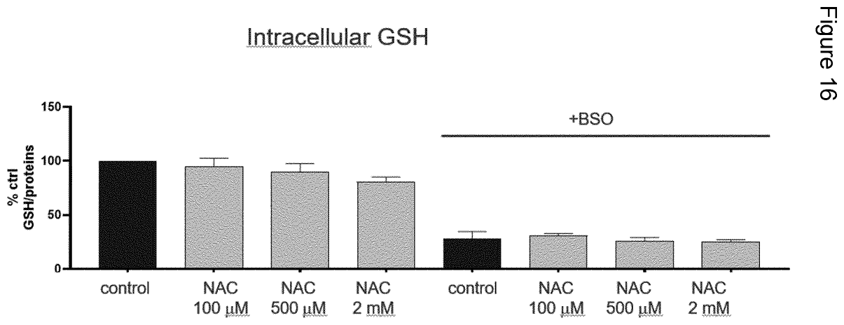

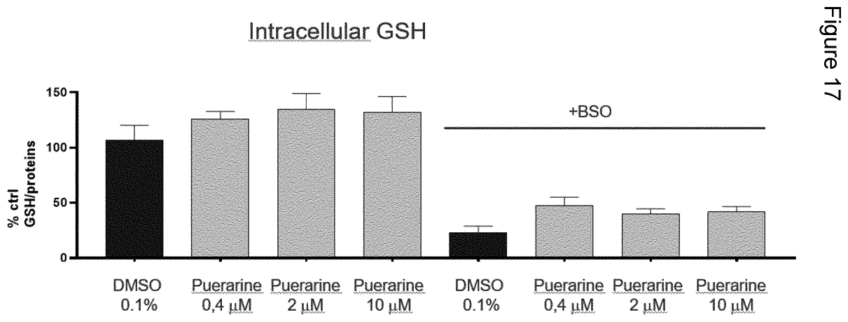

FIG. 15 shows that Ergothioneine significantly activates Nrf2 at 0.5 mM with up to an 18% increase Nrf2 levels in the cell nucleus.

### Example 5: Measurement of Intracellular Glutathione (GSH) in Rat Primary Astrocyte Culture

Intracellular GSH was measured in rat primary astrocytes in culture by using a GSH-Glo™ Assay, which is a luminescent-based assay for the detection and quantification of reduced and/or total glutathione levels in cells. The assay converted luciferin derivatives into luciferin in the presence of GSH. The reaction was catalyzed by a glutathione S-transferase (GST) enzyme supplied in the kit. The luciferin formed was detected in a coupled reaction using Ultra-Glo™ Recombinant Luciferase that generated a glow type luminescence that was proportional to the amount of glutathione present in cells. A standard curve was used for each biological replicate and at least 4 technical replicates were done for each condition and for each biological replicate. 10% of lysate of each technical and biological replicate was used to measure protein amount by BCA and used to normalize the GSH intracellular content to total proteins. Buthionine sulfoximine (BSO) a specific inhibitor of γ-glutamylcysteine ligase (GCL) was added at 15 mM at the time of the start of the treatment to confirm the specificity of the readout. Measurements were performed 48 hrs after treatment. Results were compared to the control condition for each biological replicate and for each condition.

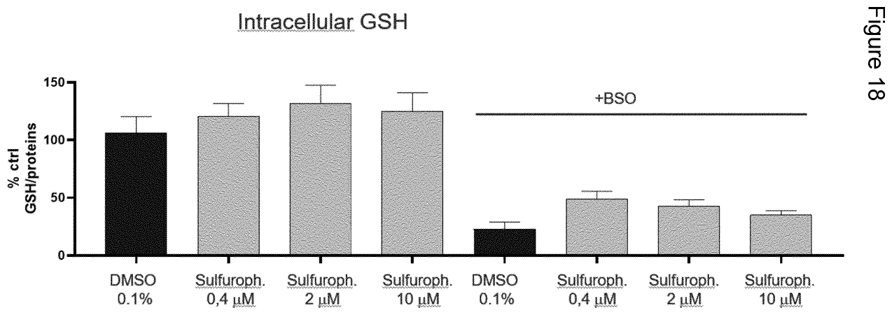

FIG. 16 shows that N-Acetylcysteine does not increase intracellular glutathione due to the culture conditions (no cysteine depletion). This is contrasted with FIG. 23 which shows that when the medium is reduced in cysteine and methionine then both N-Acetylcysteine and L-cystein increase glutathione levels.

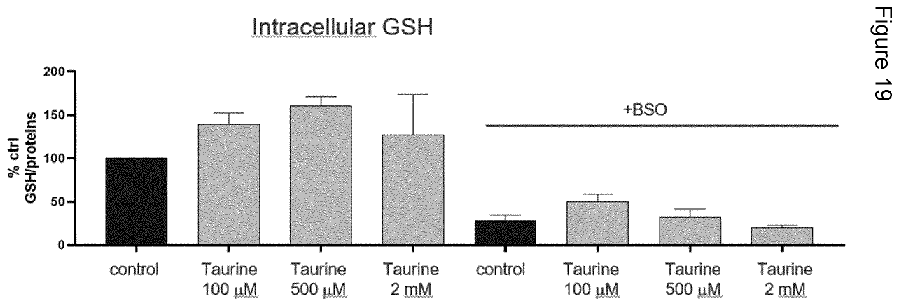

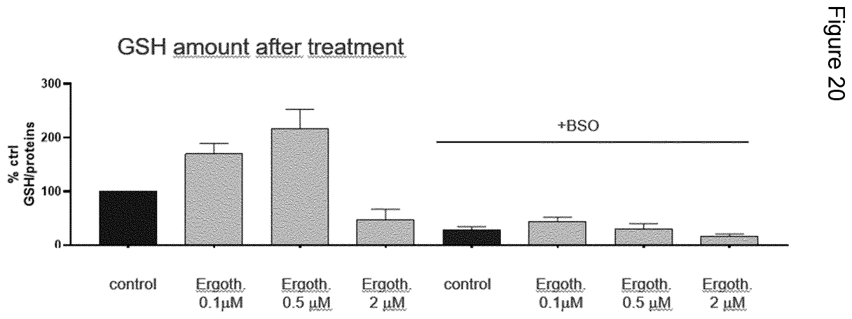

FIG. 17 demonstrates that Puerarine increases intracellular glutathione.

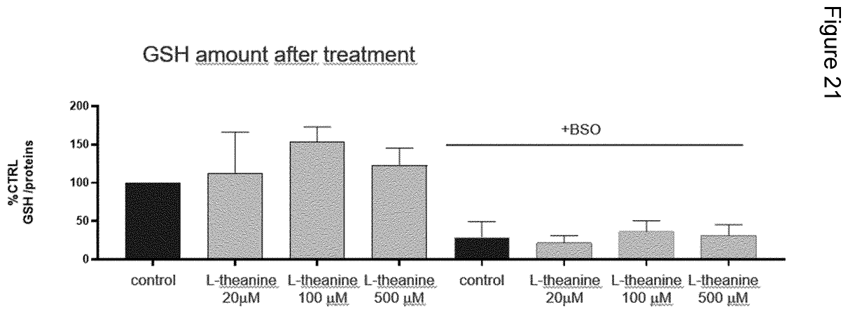

FIG. 18 demonstrates that Sulfurophane increases intracellular glutathione.

FIG. 19 demonstrates that Taurine increases intracellular glutathione.

FIG. 20 demonstrates that Ergothioneine increases intracellular glutathione.

FIG. 21 demonstrates that L-theanine increases intracellular glutathione.

Examples 3, 4 and 5 demonstrate that the various different compounds of the invention have different complementary mechanisms of action which surprisingly work together to increase glutathione.

### Example 6—Enhancement of Glutathione (GSH) Via Systemic Treatment with N-Acetyl Cysteine (NAC) Results in Increased Motivational Performance in Adult Male Rats

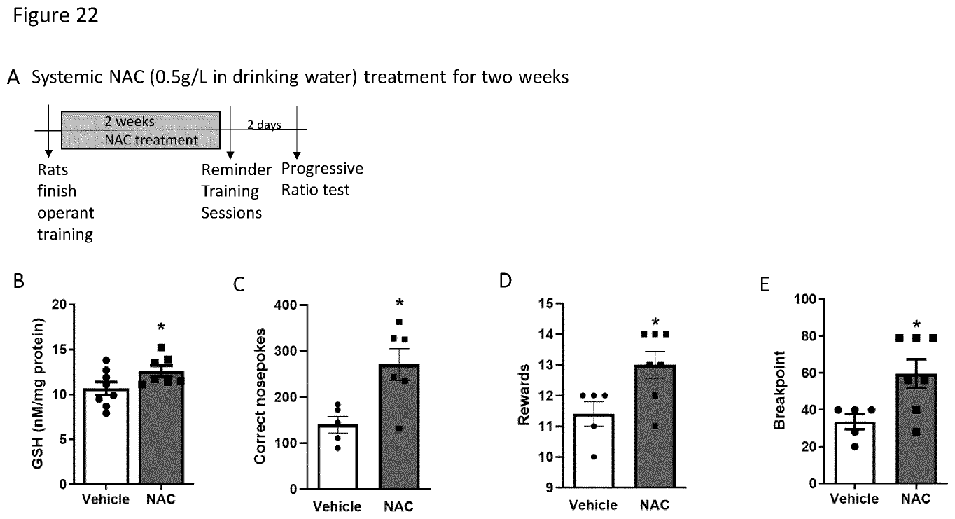

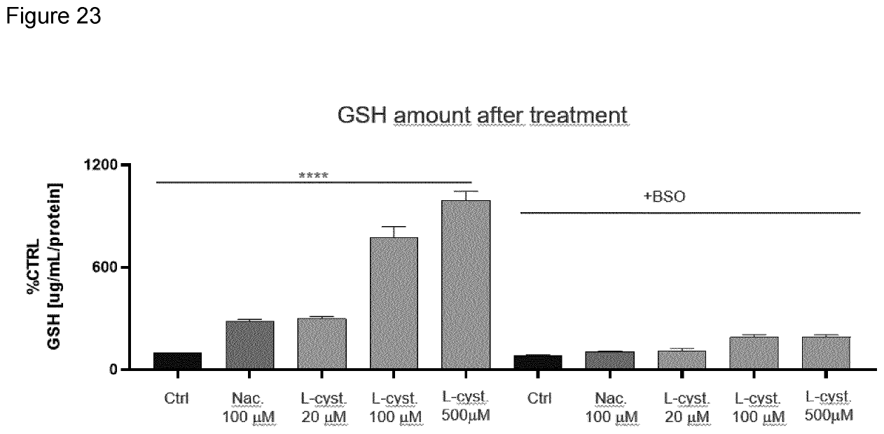

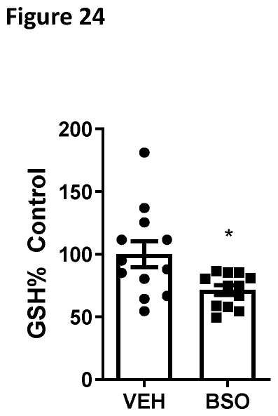

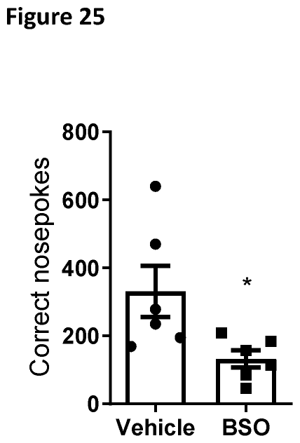

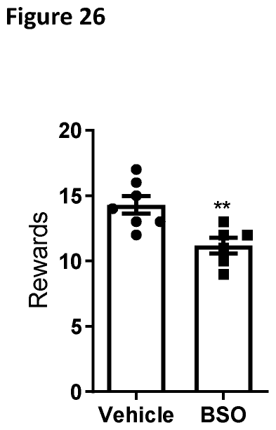

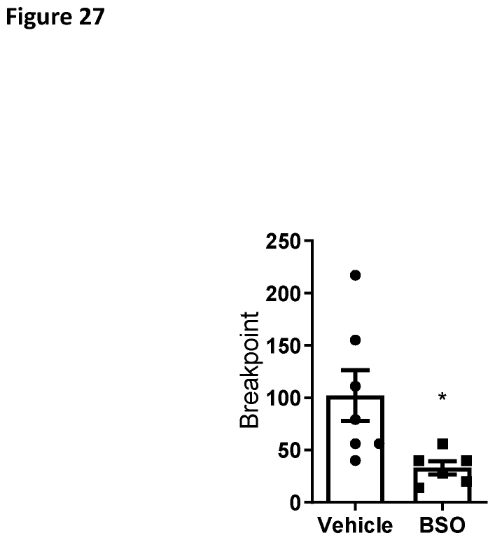

Adult male outbred rats were trained for nose poke for saccharine food pellets for one week on an FR1 training schedule as described in the methods below. Rats were then treated with either normal drinking water (vehicle) or N-acetyl cysteine (NAC) in the drinking water (FIG. 22A) at a dose that was previously shown to enhance GSH levels (FIG. 22B). Following 2 weeks of treatment, rats were given an additional 2 training sessions, followed 24 h later by a progressive ratio session designed to test their motivated behaviour. During this progressive ratio session, rats have to increasingly work harder to earn the saccharine pellet, as described in the methods below. NAC-treated rats made significantly more nose pokes during this session (FIG. 22C) and received a greater number of rewards (FIG. 22D). Finally, NAC-treated rats exhibited a significantly higher breakpoint level compared to vehicle-treated counterparts (FIG. 23E). The breakpoint is defined as the last step in the session where the animals received a reward and is a direct correlate of their willingness to exert an effort. A higher breakpoint indicates that the animal exerted greater effort during the session.

### Materials and Methods:

Animals: Adult male Wistar rats (Charles Rivers, Saint-German-Nuelle, France) weighing 250-275 gr at the beginning of the experiment were used for all experiments. Rats were individually housed in cages in housing colonies on a 12:12 h reversed light:dark cycle with lights on at 20:00, and lights-off at 8:00. Food and water were available ad libitum. Following a week of acclimatization to the animal facilities, rats were handled for 2 min per day for three days prior to the start of the experiments, in order to habituate to the experimenters.

Operant conditioning: Ten days after introduction to the reversed day-night cycle, rats started training in a fixed ratio 1 reinforcement schedule (FR1). Operant chambers (Coulbourn Instruments, Holliston, Mass., US), placed in sound attenuating cubicles, were equipped with a grid, underneath which a tray with standard bedding material was placed for collection of feces and urine after each training session. Each chamber had one food tray and two ports placed on either side of the tray. A cue light was placed in each port and the food tray, whereas a house light was placed above the food tray. The right-hand side port of each chamber was designated as “active”, meaning that spontaneous nosepoking would result in the drop of one 45 mg food pellet (Bio-Serv, Flemington, N.J., USA) to the food tray. Upon nosepoking in the active port, the cue and house lights were turned off, while the tray light turned on and the pellet dropped to the food tray. The two ports remained inactive for 20 s, during which nosepokes would not result in the delivery of a new pellet. Subsequently, the chamber returned to its initial condition. Each training session lasted maximally two hours or until a rat acquired 100 pellets. Each rat received six training sessions (one training on each day for five consecutive days, followed by two days without training and one training session on day 8). Only rats that finished at least two training sessions acquiring 100 pellets before the two-hour mark were used for progressive ratio experiments.

Subsequently, rats were treated with N-acetyl cysteine in the drinking water (500 mg/L) or continued having access to normal water (control). After two weeks of treatment, rats were exposed to another two days of FR1 training to ensure their training performance was similar to pre-treatment levels.

To test motivated behaviour, rats were exposed to a progressive ratio reinforcement schedule (progressive ratio test). Progressive ratio sessions were identical to training sessions except that the operant requirement in each trial (T) was the integer (rounded down) of the function 1.4^{(T-1)} starting at one nosepoke for the first three trials and increasing in subsequent trials for rats (Wanat et al. Nat Neurosci. 2013; 16(4):383-5). Progressive ratio sessions lasted two hours. Correct nosepokes (i.e. nosepokes in the active port and outside the timeout period, thus resulting in food delivery) were calculated to evaluate behavioural performance, as well as the number of acquired rewards and the last completed ratio (breakpoint).

### Example 8: GSH Increases after N-Acetyl Cysteine and L-Cysteine Administration in Medium Reduced in Cysteine and Methionine

To measure GSH increase after N-acetyl cysteine (Nac) and L-cysteine supplementation, rat primary astrocytes were cultured in medium depleted of methionine (a precursor of cysteine) and cystine but still supplemented with 15% FBS to not have a full depletion but rather a reduction of these amino acids. Experiments were performed only with the same batch of FBS to avoid any difference in amino-acid content amongst batches.

Because standard DMEM is very high in cysteine and its precursor methionine, to see a full dynamic range in response to the supplementation we used a reduction in cysteine, however, not a total depletion as cysteine is still present in FBS. FIG. 23 demonstrated that NAC and L-cysteine significantly increase the GSH intracellular level.

### Example 9: BSO an Inhibitor of Glutathione Modifies Motivational Performance

Investigation of the Nucleus accumbens was undertaken in cannulated rats which were then moved to a reverse light-dark cycle to recover for 10 days. Subsequently, they were trained in a fixed ratio 1 reinforcement schedule (FR1) for 8 days (six training sessions). Following the last FR1 session and 24 hours before a progressive ratio (PR) reinforcement schedule session, they were then infused in the Nucleus Accumbens with 1 μl of vehicle (saline) or buthionine sulfoximine (BSO) (7 μg/μl), the compound is known to reduce the levels of glutathione by inhibiting the gamma-glutamylcysteine ligase (GCL), the enzyme required in the first step of glutathione synthesis. Progressive ratio sessions were identical to training sessions except that the operant requirement in each trial (T) was the integer (rounded down) of the function 1.4(T−1) starting at one nosepoke for the first three trials and increasing in subsequent trials for them (Wanat et al. Nat Neurosci. 2013; 16(4):383-5). Progressive ratio sessions lasted two hours. Correct nosepokes (i.e. nosepokes in the active port and outside the timeout period, thus resulting in food delivery) were calculated to evaluate behavioural performance, as well as the number of acquired rewards and the last completed ratio (breakpoint). All experiments were performed with the approval of the Cantonal Veterinary Authorities (Vaud, Switzerland).

### Results

A single injection of BSO 24 hours before the test reduced GSH levels by 29% in the Nucleus Accumbens (FIG. 24). This resulted in decreased performance of the BSO-treated compared to the vehicle-treated animals in the operant conditioning paradigm (PR-schedule). This was seen by a significant reduction in correct nosepokes (−60%) shown in FIG. 25, rewards (−22%) shown in FIG. 26 and breakpoint (−68%) shown in FIG. 27.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method increasing glutathione levels in the brain comprising administering a composition to an individual in need of same wherein said composition comprises at least one compound selected from the group consisting of:
(i) compounds which are substrates or precursors of substrates of glutathione synthesis;
(ii) compounds which are targeting the regulation of anti-oxidant expression levels of glutathione by targeting Nrf2-dependent regulation and
(iii) compounds which further potentiate the antioxidant effect of glutathione.

2. Method according to claim 1 wherein glutathione is increased in the nucleus accumbens region of the brain.

3. Method according to claim 1 wherein said compounds of group (i) which are substrates or precursors of substrates of glutathione synthesis are selected from the group consisting of: glycine, cysteine, glutamate, N-Acetylcysteine, taurine, and whey proteins, and their functional derivatives.

4. Method according to claim 1 wherein said compounds of group (ii) which are targeting the regulation of anti-oxidant expression levels of glutathione by targeting Nrf2-dependent regulation are selected from the group consisting of: sulforaphane, dimethylfumarate, curcumin, melatonin and trehalose and/or their functional derivatives.

5. Method according to claim 1 wherein said compounds of group (iii) which further potentiate the antioxidant effect of glutathione are selected from the group consisting of: puerarine, ergothioneine, l-carnitine, l-thionine and/or glutamine and their functional derivatives.

6. Method according to claim 1 wherein said composition comprises N-acetylcysteine for use in increasing motivational performance or mental energy.

7. Method according to claim 6 wherein said composition comprises N-acetylcysteine and glycine for use in increasing motivational performance or mental energy.

8. Method according to claim 1 wherein said composition comprises N-acetylcysteine, puerarine, sulforaphane for use in increasing motivational performance or mental energy.

9. Method according to claim 1 administered with additional glutathione for use in increasing motivational performance or mental energy.

10. Method according to claim 1 wherein said composition is administered orally.

11. Method according to claim 1 wherein said composition is formulated as a composition selected from the group consisting of a food product, a food for special medical purposes (FSMP), a nutritional supplement, a ready to drink formula, a dairy-based drink, a low-volume liquid supplement, powder formats for liquid reconstitution, a meal replacement beverage, and combinations thereof.

12. Method according to claim 1 administered together with dietary recommendations for a diet rich in glutathione comprising foods selected from the group consisting of: (a) cruciferous vegetables: broccoli, cauliflower, Brussels sprouts, and bok choy; (b) allium vegetables: garlic and onions; (c) eggs, nuts, legumes, fish, and chicken and/or (d) glutathione-rich herbs: milk thistle, flaxseed, guso seaweed.

13. Method according to claim 1 administered together with lifestyle recommendations to get at least 6 hours of sleep per night.

14. Method of claim 1 wherein the individual is a healthy individual in need of increasing motivational performance and/or mental energy.

15. Method of claim 1 wherein the individual is a healthy individual in need of increasing cognitive performance.

16. Method of claim 1 wherein the individual is a healthy individual in need of increasing motor performance.

17. Method of claim 1 wherein the individual suffers from low glutathione levels.

18. Method of claim 1 for use in a method of treatment to decrease performance anxiety.

19. Method of claim 1 for use in a method of treatment to decrease stress.

20. A method of improving motivational performance and/or mental energy in a healthy subject by administration of a glutathione enhancing composition comprising at least one compound selected from the group consisting of:
(i) compounds which are substrates or precursors of substrates of glutathione synthesis;
(ii) compounds which are targeting the regulation of anti-oxidant expression levels of glutathione by targeting Nrf2-dependent regulation and
(iii) compounds which further potentiate the antioxidant effect of glutathione.

21. (canceled)
